(19) United States
(12) Patent Application Publication
(10) Pub. No.: US 2019/0001122 A1
(43) Pub. Date: Jan. 3, 2019

(54) LEAD FOR NEUROMODULATION

(71) Applicant: G-Thereapeutics BV, Eindhoven (NL)

(72) Inventors: Damien GANTY, Eindhoven (NL); Bert BAKKER, Eindhoven (NL); Vincent DELATTRE, Eindhoven (AT); Sjaak DECKERS, Eindhoven (NL)

(21) Appl. No.: 15/989,598

(22) Filed: May 25, 2018

(30) Foreign Application Priority Data
Jun. 30, 2017 (EP) 17178941.5 - 1666

(51) Int. Cl. A61N 1/05; A61B 5/04; A61B 5/00; A61N 1/36

(52) U.S. Cl. CPC A61N 1/0553; A61B 5/04001; A61B 2562/0209; A61N 1/0558; A61N 1/36062; A61B 5/407

(57) ABSTRACT

The present disclosure relates to a lead for neuromodulation and/or neurosensing, more in particular for neurostimulation and/or neurorecording, comprising at least one lead cable, at least one electrode section and at least one electrode placed in the electrode section, wherein the lead comprises an anchor, which are pre-attached to the lead (and configured to engage with tissue fixation instrument and/or hold a tissue fixation.

### CROSS-REFERENCE TO RELATED APPLICATION

This application claims priority to European Patent Application No. 17178941.5-1666, entitled “A LEAD FOR NEUROMODULATION,” filed on Jun. 30, 2017, the entire contents of which is incorporated herein by reference in its entirety for all purposes.

### BACKGROUND AND SUMMARY

The present disclosure relates to a lead for neuromodulation and/or neurosensing, more particularly for neurostimulation and/or neurorecording, comprising at least one lead cable, at least one electrode section and at least one electrode placed in the electrode section.

Implantable leads with electrodes for spinal cord stimulation are known. In spinal cord stimulation, such electrodes are implanted into the spinal canal and are more or less directly attached to the spinal cord. To provide the neurostimulation to the spinal cord in the planned manner, it is important to reach the correct implantation position and to fix or anchor the lead and the electrodes at this position.

It appears that existing anchoring techniques are not easy to establish in this context. In particular it has been reported that the lead might move when placing the anchor on the lead body. Alternatively, existing anchors do not allow fixing the lead properly or are not stable. Also, sliding of anchors along the lead body has been observed.

Fixed anchors are already used for leads but such anchors do not form a convenient solution because of their shape, size, and specific location. Such fixed anchors may be established by small silicone parts, which are glued or overmolded on the lead body with wings and holes for a suture or the like.

It has also been reported that neurosurgeons in rare cases used the lead body for fixing sutures, thereby creating punctures in the lead body. Such punctures are, however, undesirable.

As an alternative to the above solutions, U.S. Pat. No. 7,099,718 B1 relates to a neural stimulation lead fixation. The implantable lead has at least one electrode contact at or near its distal end that prevents undesirable movement of the electrode contact from its initial implant location. One embodiment relates to a spinal cord stimulation (SCS) lead. A balloon may be positioned on the electrode lead array. The balloon is filled with air, liquid or a compliant material. When inflated, the balloon stabilizes the lead with respect to the spinal cord and holds the lead in place. The pressure of the balloon is monitored or otherwise controlled during the filling process in order to determine at what point the filling process should be discontinued. An elastic aspect of the balloon serves as a contained relief valve to limit the pressure the balloon may place on the surrounding tissues when the epidural space is constrained.

It is therefore an object of the present disclosure to provide an improved lead for neuromodulation and/or neurosensing, particularly in that the lead may be fixed at the implantation site with high accuracy and that the fixation is easy to establish.

This object is solved according to the present disclosure by a lead for neuromodulation and/or neurosensing. More specifically, a lead for neuromodulation and/or neurosensing is provided, in particular a lead for neurostimulation and/or neurorecording, which comprises at least one lead cable, at least one electrode section and at least one electrode placed in the electrode section, wherein the lead comprises an anchoring section or anchor, which is pre-attached to the lead and configured to engage with tissue fixation means, also referred to herein as “tissue fixation instrument”, and/or hold a tissue fixation.

The lead may be a medical lead and it may be designed to be positioned for long-term implantation.

The lead cable of the lead may be, for example, an integral part and directly connected to the electrode section. The electrode section may be formed by one electrode at the tip end of the lead cable. In another example, the electrode section may be part of a paddle with a plurality of electrodes, together forming the electrode section.

The lead cable may in some examples be detachable or may be a kind of extension cable.

Further, the lead cable may be formed just by a short connector being connected to the electrode section, wherein the connector is configured and arranged to be connected with an extension cable (e.g. for connection with another medical device and/or a controller and/or an implantable pulse generator (IPG)).

Specifically, the lead may be a lead for spinal cord neurostimulation and designed to be placed in the spinal canal, more specifically in the epidural or in the subdural space.

The tissue fixation with the tissue fixation means or tissue fixation instrument are selected from at least one of suture, suture fiber, clamp, bracket, staples or the like.

The anchoring means, also referred to herein as “anchor” may be arranged in or within or close to the electrode section. For example, the anchoring means may be established by a hole within the paddle of the lead, such that, for example, sutures and/or clamps may be engaged with such holes.

Still further, the anchoring means may be positioned or arranged on or around at least one lead cable.

The present disclosure is based on the basic idea that by pre-attaching at least one anchoring section to the lead, the position relative between lead and the anchoring means is already defined and set. Thus, no relative movement during surgery of the lead and the anchoring means is possible. Moreover, the position of the anchoring means may be used as additional guiding means for the surgeon, as the anchoring means may still be at least partially accessible or at least partially outside of the spinal canal and so may be used as an orientation means for placement of the lead. In particular the anchoring means may be arranged and configured to be capable to receive punctures for engaging and/or holding the fixation means. For example, the anchoring means may be flexible and/or elastic.

Moreover, in one example the anchoring section may be a sleeve or sleeve-like section on the outer part of the lead cable, which may for example form an outmost part of the lead cable.

The anchoring means may be overmolded over and/or part of the outer part of the lead cable. By means of overmolding a simplified pre-attachment of the anchoring means to the lead may be provided. If the anchoring means is part of the outer part of the lead cable, it may be desirable to create such a section, for example, by varying the material thickness or by two or more component molding processes.

The anchoring means may be at least partially made of a thermoplastic material, in particular a medical grade thermoplastic material, for example poly amide or poly ethylene polyurethane.

The anchoring means may be at least partially made of an elastomeric material, especially a medical grade elastomeric thermoplastic material, for example medical grade silicone.

By this, punctures to the anchoring means for engaging with the fixation means like a suture or clamp or the like can be easily made and held by the anchoring means. Furthermore, such material may be on the one hand suitable to be implanted even for long-term applications and on the other hand mechanically stable enough to maintain the engagement with the fixation means over the life-time of the lead.

Furthermore, in one example the anchoring section may have an uneven outer side. Such an uneven outer side may be formed by or with bumps, indentations or the like. Such bumps or indentations may be additionally used to keep wires and/or fiber of the suture in place, so that they may not move away. Bumps or indentations may also be used to be targeted by and be engaged with specifically clamps or the like.

The outer side of the anchoring section may be at least partially substantially pearly and/or comprise recesses and ridges. Such a structure may be readily formed, for example, by a molding process. Also, such as structure may be used to keep wires and/or fiber of the suture in place, so that they may not move away.

Additionally, in some examples there may be at least two anchoring means. Thus, in this regard more options for fixation may be provided to the surgeon. Further, both (in the case of two) anchoring means may be used to enhance the fixation of the overall lead. It may, however, also be envisioned that the fixation may be sufficient with at least one engagement of one anchoring means with fixation means and that the one anchoring means is chosen by the surgeon, as to which is better or more readily accessible out the of the at least two anchoring means.

Moreover, in a further embodiment the anchoring means close to the electrode section may be at a distal part of the lead cable. By such a layout the anchoring means may be close to the electrode section and may therefore block and/or minimize unwanted movements and rotations of the electrode section. Thus, a correct or desired positioning for the electrode section may be established. Also, this may help to maintain the electrode positioning over a long time, for example over the complete lifetime of the implanted lead.

There may be an anchoring means at a proximal part of the lead cable. Such an anchoring means may be used to fix the lead cable in a position close to, for example, an implantable pulse generator or any other element that may be connected to the lead. This may be realized in some examples by providing a second anchoring means or by providing an elongated anchoring means, for example extending from a distal part of the lead cable to a proximal part of the lead cable.

Also, there may be between the anchoring means close to the electrode section and the electrode section a free lead cable section without an anchoring section. This section may be used for freely forming and moving the lead cable in this part of the lead. In this way, the possibilities or options for steering and moving of the lead may be enhanced or improved.

Moreover, the free lead cable section may have a minimum length chosen out of a range of approx. 5 mm, or more generally a length of chosen out of a range of approx. 10 mm to 60 mm This range or ranges of the length for the free lead cable section may be advantageous for enhancing the options for steering and moving of the lead and at the same time having the anchoring section still close to the electrode section for providing a stable anchoring and fixation for/of the electrode section.

Additionally, the anchoring section may have a length of at least approx. 10 mm or more, or in some examples, approx. 60 mm or more.

Furthermore, the lead may comprise a strain relief loop. Such a strain relief loop may be formed by a loop of one of the lead cables. It may for example comprise less flexibility than other parts of the lead cable. The stiffening may be achieved and provided by the anchoring means.

The strain relief loop may in some examples be pre-formed and/or pre-shaped.

This may be achieved by means of the anchoring means. In particular, the anchoring means may be stiff enough (but, for example, still formable) to form the strain relief loop.

Thus, the anchoring means may be pre-attached to the lead cable or more generally to the lead cable, i.e. such that the strain relief loop is already present when the lead is lying on a static position.

By means of the strain relief loop the application of strain and tension in the lead cable distal of the strain relief loop may be avoided or minimized In this way, strain and tension may not be forwarded to the electrode section, which may help maintain a correct or desired position of the electrodes.

The lead may be a spinal cord neurostimulation lead, in particular wherein the electrode section is an electrode paddle with an array of electrodes, for example configured to be implanted in a vicinity of the cervical, thoracic, lumbar or sacral section of the spinal cord.

As an alternative embodiment, the anchoring means may be formed as part of the electrode section or as at least one hole in, for example, the electrode section of the lead.

The electrode section of the lead may comprise at least one anchoring means and/or may be directly connected to at least one anchoring means.

The anchoring means may be embodied as an extra lead part pre-attached to the electrode section.

The electrode section may be, for example, embodied as an electrode paddle.

The anchoring means may be not be attached to the lead cable but may be directly connected to the electrode section, for example the electrode paddle. The attachment of the anchoring means to the electrode section may be done by molding, overmolding, or gluing, or by forming a further integral part of the electrode section, for example by providing an extension or finger-like or wing-like or outgrowing part or paddle-like part, which may serve as anchoring means.

The anchoring means may be made of semi-rigid materials (non-metallic except for the screw or the fixation means) such as a thermoplastic and/or elastomeric material and may be additionally or alternatively in a sleeve-like or half-sleeve form with wings for any suitable fixation means.

The anchoring means may have one or more hole(s) to be attached with a suture fiber, a screw, a pin, clamp or any other suitable fixation means.

The anchoring means may not be linked (attached to) current-carrying parts such as lead bodies or the lead cable in order to minimize applied constraint on electrical parts.

The present disclosure also relates to a method of fixing a lead for neuromodulation. Accordingly, a method of fixing a lead for neuromodulation is provided, wherein the lead comprises at least one lead cable, at least one electrode section and at least one electrode placed in the electrode section, wherein the lead further comprises an anchoring section, the method comprising at least the following steps:

placing the lead at the implantation site,

fixing the lead at the implantation site by means of a fixation means being engaged with the surrounding tissue of the patient and engaged with the anchoring section.

The lead may be a lead as set forth above or described hereinafter.

Further details and advantages of the present disclosure shall now be disclosed in connection with the drawings.

### BRIEF DESCRIPTIONS OF THE FIGURES

FIG. 1 shows a perspective view of a first embodiment of a lead according to the present disclosure.

FIG. 2 shows a perspective view of a second embodiment of a lead according to the present disclosure.

FIG. 3 show a perspective view of a third embodiment of a lead according to the present disclosure.

FIG. 4 shows a perspective view of a fourth embodiment of a lead according to the present disclosure.

FIG. 5 shows a perspective view of a fifth embodiment of a lead according to the present disclosure.

FIG. 6 shows a further view of a sixth embodiment of a lead according to the present disclosure.

FIG. 7 shows a further view of the embodiment of FIG. 6.

FIG. 8 shows a further view of a seventh embodiment of a lead according to the present disclosure.

FIG. 9 shows a flow chart of an example procedure of fixation of the lead.

### DETAILED DESCRIPTION

FIG. 1 shows a perspective view of first embodiment of a lead 10 according to the present disclosure.

The lead 10 is a lead for neuromodulation and neurosensing, more in particular for neurostimulation and neurorecording.

The lead 10 comprises two lead cables 12, 14 and an electrode section 16 arranged on an electrode paddle 18.

Specifically, the lead 10 is a spinal cord neurostimulation lead.

The electrode paddle 18 is made of flexible material, for example comprising a silicone based material.

The electrode section 16 comprises three rows of electrodes 20, having an inner electrode row 22 in the middle of the electrode paddle 18 and two outer electrode rows 24, 26.

The electrode paddle 18 with the array of electrodes 20 is configured to be implanted in a vicinity of a lumbar section of a spinal cord, for example to be placed in a spinal canal.

In another example, such an electrode array or a similar electrode array may be used to be implanted in a cervical, thoracic, or sacral section of the spinal cord

Furthermore, the lead 10 comprises an anchoring means 28, which is pre-attached to the lead 10 and which is configured to engage with tissue fixation means (not shown) and hold a tissue fixation.

The tissue fixation with the tissue fixation means are selected from at least one of suture, suture fiber, clamp, bracket, staples or the like.

The anchoring means 28 is here a sleeve-like section on the outer part of the lead cable 14, here forming an outermost part of the lead cable 14.

Moreover, the anchoring means 28 are overmolded over and part of the outermost part of the lead cable 14.

The anchoring means 28 may at least partially be made of a thermoplastic material, especially a medical grade thermoplastic material, for example poly amide or poly ethylene polyurethane.

In another example, the anchoring means 28 is at least partially made of an elastomeric material, namely a medical grade elastomeric thermoplastic material, for example medical grade silicone.

Furthermore, the anchoring means 28 has a length 21. As an example, anchoring means 28 may have a length of approx. 100 mm or more, in some examples approx. 60 mm or more.

As can be seen in FIG. 1, the anchoring means 28 comprises an uneven outer side, i.e. it is pearly and comprises recesses 28a and ridges 28b. Also, it may be described as having a plurality of bumps, here identically shaped bumps 28b.

Between the anchoring means 28 (close to the electrode section 16 and the electrode paddle 18) and the electrode section is a free lead cable section 30 without anchoring means.

The free lead cable section 30 has a length of approx. 20 mm. As examples, the free lead cable section may have a minimum length selected from a range of approx. 5 mm, more in particular a length selected from a range of approx. 10 mm to 60 mm.

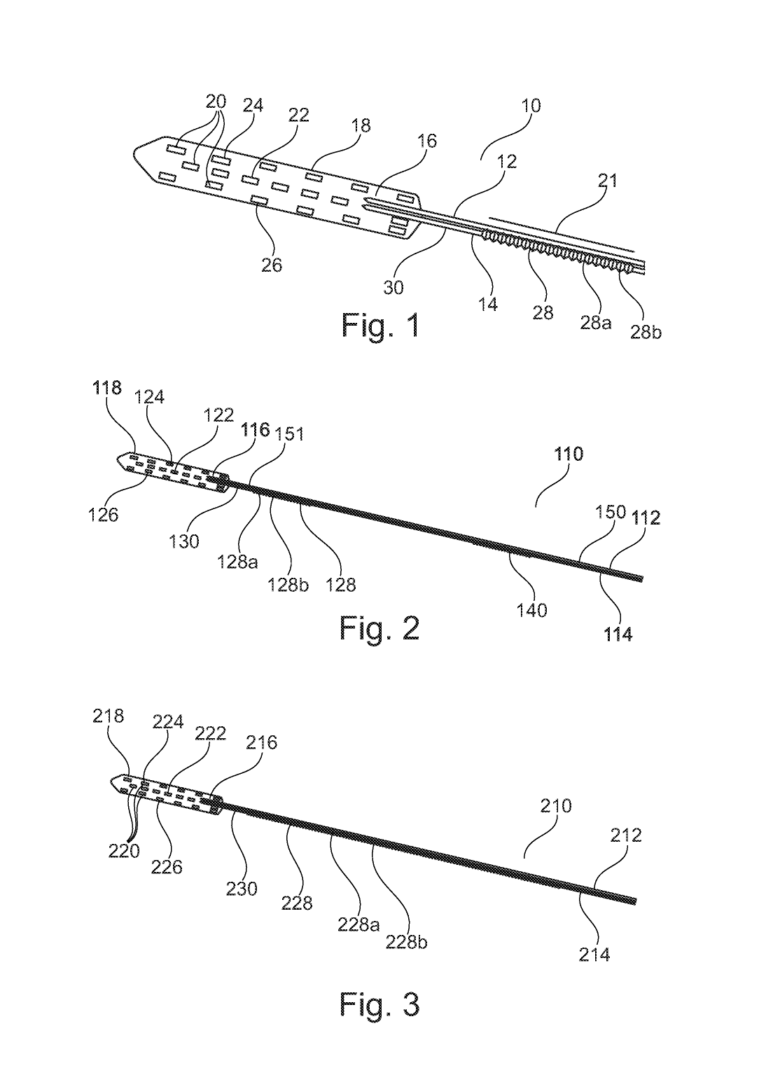

FIG. 2 shows a perspective view of a second embodiment of a lead 110 according to the present disclosure.

The lead 110 comprises each and every structural and functional feature as shown in FIG. 1 and as described above.

Similar or identical features have the same reference number, however increased by the value 100.

As an additional feature, the lead 110 comprises a second anchoring means or section 140.

The second anchoring section 140 is spaced apart from the first anchoring means 128 on the same lead cable 114.

The second anchoring means 140 is at the proximal part 150 of the lead cable 114 while anchoring means 128 is at the distal part 151 of the lead cable, so that the lead cable 114 may be anchored in a section close to a connection to an implantable pulse generator (IPG). Discussed herein, it may be understood that distal refers a part of the lead cable closer to the electrode section, while proximal refers to a part of the lead cable closer to the IPG.

FIG. 3 shows a perspective view of a third embodiment of a lead 210 according to the present disclosure.

The lead 210 comprises each and every structural and functional feature as shown in FIG. 1 and as described above.

Similar or identical features have the same reference number, however increased by the value 200.

As an additional feature, the lead 110 comprises an elongated anchoring means 228, which covers more than ⅔ of the length of the lead cable 214. Thus, the same anchoring means 228 may be used for a fixation close to the proximal part and the distal part of the lead cable 214.

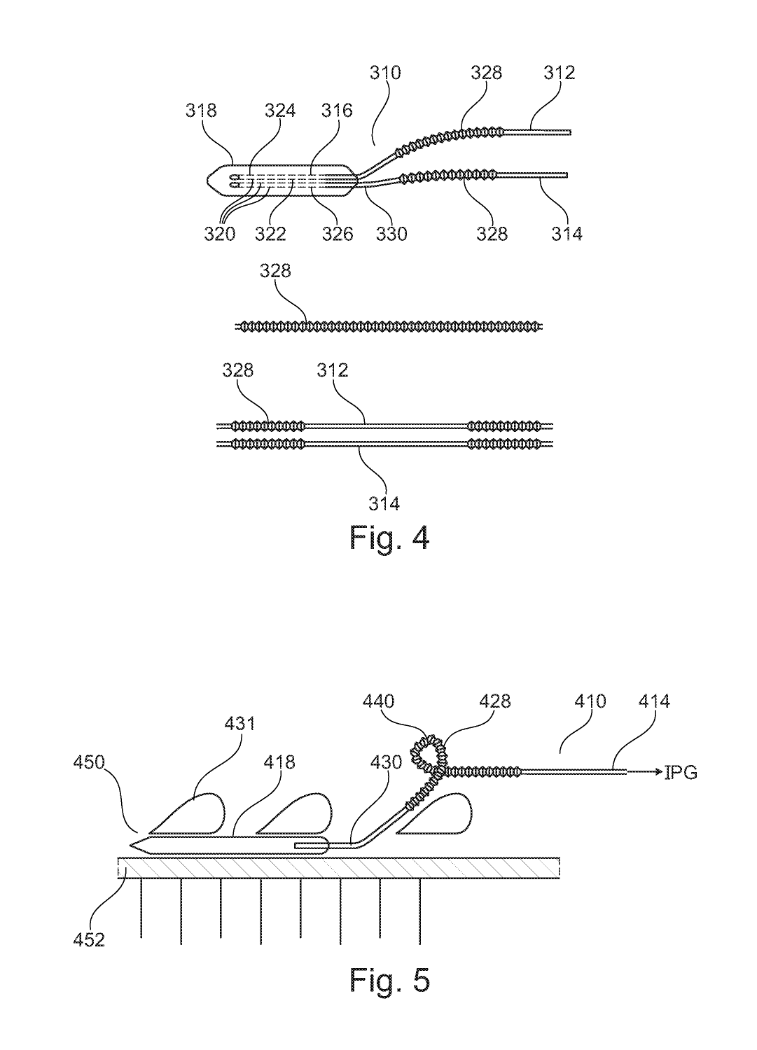

FIG. 4 shows a perspective view of a fourth embodiment of a lead 310 according to the present disclosure.

The lead 310 comprises each and every structural and functional feature as shown in FIG. 1 and as described above.

Similar or identical features have the same reference number, however increased by the value 300.

As an additional feature, the lead 310 comprises anchoring means 328 at each lead cable in the distal area of lead cables 312 and 314.

It is also possible, that there is on both lead cables a second proximal anchoring means likewise the second anchoring means 140.

FIG. 5 shows a perspective view of a fifth embodiment of a lead 410 according to the present disclosure.

The lead 410 comprises each and every structural and functional feature as shown in FIG. 1 and as described above.

Similar or identical features have the same reference number, however increased by the value 400.

As an additional feature, the lead 410 comprises a strain relief loop 440.

The whole strain relief loop 440 is covered with the anchoring means 428.

The function of the strain relief loop is to allow application of strain and tension in the lead cable 414 distal of the strain relief loop 440, so that strain and tension are not forwarded to the electrode paddle 418.

The electrode paddle 418 is here shown in FIG. 5 as being placed in the spinal canal 450 close to the lumbar spinal cord 452.

As can be also seen, the strain relief loop 440 is outside of the spinal canal 450 and also outside of the vertebrae 431. So, a fixation can be done with the tissue surrounding the vertebrae at this location.

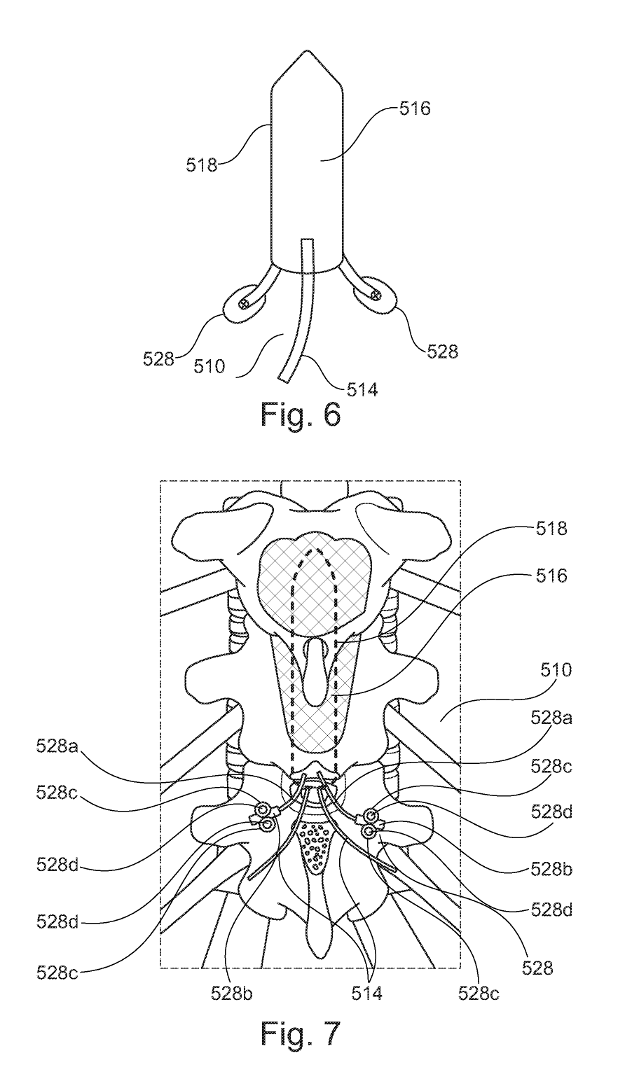

FIG. 6 and FIG. 7 show a sixth embodiment of a lead 510 according to the present disclosure.

FIG. 7 shows the implanted lead 510 in an implantation site of a human vertebrae and spinal cord at a lumbar part of the spinal cord.

The lead 510 comprises each and every structural and functional feature as shown in FIG. 1 and as described above.

Similar or identical features have the same reference number, however increased by the value 500.

As additional features, the lead 510 comprises instead of anchoring means attached to the lead cable(s) two paddle anchoring means 528, which are directly attached and connected to the electrode section 516, here being formed on and arranged on the electrode paddle 518.

The two paddle anchoring means 528 are arranged here on both sides of the lead cable 514 or lead cables 514 (as shown in FIG. 7) at the proximal end or proximal part of the electrode paddle 518.

Moreover, the paddle anchoring means 528 is not attached to the lead cables 514 but only and directly connected to the electrode paddle 518.

Thus, the paddle anchoring means 528 are not linked (attached to) current-carrying parts such as lead bodies or the lead cable(s) in order to minimize applied constraint on electrical parts.

The attachment of the anchoring means to the electrode section may be done by molding, overmolding, gluing, or by forming a further integral part of the electrode section, for example by means of providing an extension or finger-like, wing-like, or outgrowing part or paddle-like part, which serves as anchoring means.

Here, at FIG. 7 anchoring means 528 are formed sleeve-like on a paddle-extension cable 528a as having a sleeve 528b with two wings 528c.

In each wing 528c there is a hole 528d to be attached with a suture fiber or a screw or a pin or clamp or any other suitable fixation means.

The paddle anchoring means 528 may be made of semi-rigid materials (non-metallic except for the screw or the fixation means) such as a thermoplastic and/or elastomeric material and may be additionally or alternatively in a sleeve like or half sleeve form with wings for any suitable fixation means.

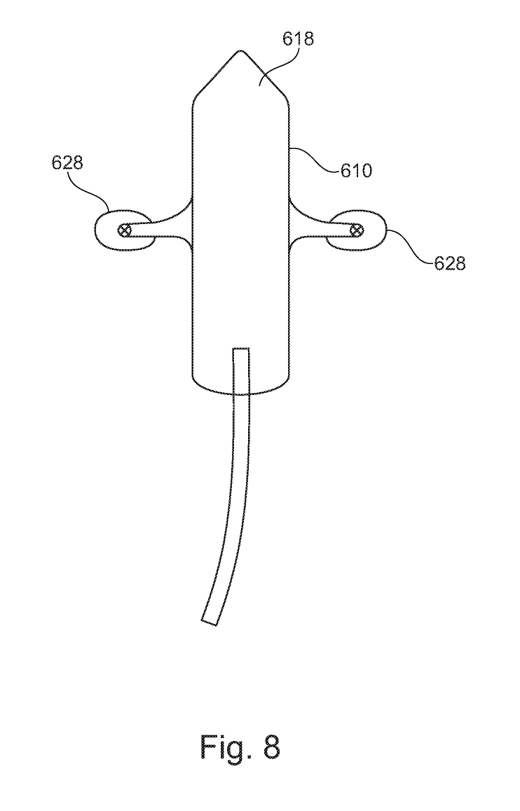

FIG. 8 shows a seventh embodiment of a lead 610 according to the present disclosure.

The lead 610 comprises each and every structural and functional feature as shown in FIG. 6 and FIG. 7 and as described above.

Similar or identical features have the same reference number, however increased by the value 100 compared to the reference number of FIG. 6 and FIG. 7.

The only difference is the fact that the paddle anchoring means 628 are arranged in a middle section at the sides of the electrode paddle 618.

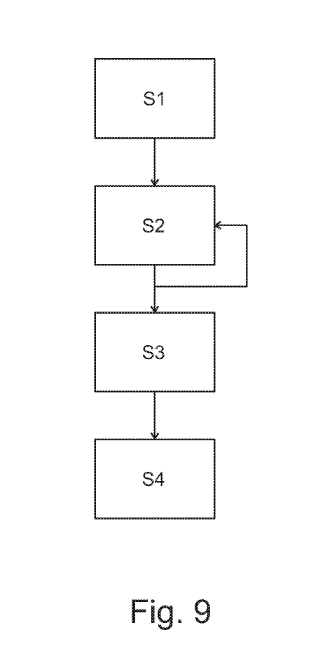

FIG. 9 shows a flow chart of an example procedure of fixation of the lead, here the lead 10.

The fixation of the lead 10 comprises the following steps:

Firstly, in step S1, the lead 10 is placed at the implantation site by a surgeon. Here, the surgeon positions the lead 10 with the electrode paddle 18 in the spinal canal, for example in a vicinity of a lumbar section of the spinal cord.

In the second step S2 the electrodes 22, 24, 26 and thus the placement of the electrode section 16 is controlled, for example, by several testings. For example, test stimulation patterns can be used and the response thereto checked. There may be iterations of this step. If the test confirms correct placement, then it is continued with third step S3.

By performing some tests in step S2 the correct and intended placement and position of the lead 10 with the electrode paddle 18 is confirmed.

Then, in the third step S3, the lead 10 is fixed at the implantation site by means of a fixation means being engaged with the surrounding tissue of the patient and engaged with the anchoring means 28.

For example, this can be done by engaging a suture with the anchoring means 28 and surrounding tissue. The suture fiber may then be wound around the bumps 28b and may be held in the recesses 28a.

In the fourth step S4 then the surgery is completed, i.e. by continuing with the implantation of other elements of an implantable medical system and/or finalization of the surgical intervention.

### REFERENCES

10 lead
12 lead cable
14 lead cable
16 electrode section
18 electrode paddle
20 electrode
22 inner electrode row
24 outer electrode row
26 outer electrode row
28 anchoring means
28a recess
28b ridge, bump
30 free lead cable section
110 lead
112 lead cable
114 lead cable
116 electrode section
118 electrode paddle
120 electrode
122 inner electrode row
124 outer electrode row
126 outer electrode row
128 anchoring means
128a recess
128b ridge, bump
130 free lead cable section
140 second anchoring means
210 lead
212 lead cable
214 lead cable
216 electrode section
218 electrode paddle
220 electrode
222 inner electrode row
224 outer electrode row
226 outer electrode row
228 anchoring means
228a recess
228b ridge, bump
230 free lead cable section
310 lead
312 lead cable
314 lead cable
316 electrode section
318 electrode paddle
320 electrode
322 inner electrode row
324 outer electrode row
326 outer electrode row
328 anchoring means
328a recess
328b ridge, bump
330 free lead cable section
410 lead
412 lead cable
414 lead cable
416 electrode section
418 electrode paddle
420 electrode
422 inner electrode row
424 outer electrode row
426 outer electrode row
428 anchoring means
428a recess
428b ridge, bump
430 free lead cable section
440 strain relief loop
510 lead
514 lead cable
516 electrode section
518 electrode paddle
528 anchoring means
528a paddle-extension cable
528b sleeve
528c wing
528d hole
S1 step 1 of fixation method
S2 step 2 of fixation method
S3 step 3 of fixation method
S4 step 4 of fixation method

1. A lead for neuromodulation and/or neurosensing comprising at least one lead cable, at least one electrode section and at least one electrode placed in the electrode section, wherein the lead comprises an anchor, which is pre-attached to the lead and configured to engage with a tissue fixation apparatus and/or hold a tissue fixation.

2. The lead according to claim 1, wherein the anchor is a sleeve or sleeve-like section on an outer part of the lead cable, where the sleeve or sleeve-like section forms an outermost part of the lead cable.

3. The lead according to claim 2, wherein the anchor is overmolded over and/or is part of the outer part of the lead cable.

4. The lead according to claim 1, wherein the anchor is at least partially made of a thermoplastic material.

5. The lead according to claim 1, wherein the anchor is at least partially made of an elastomeric material.

6. The lead according to claim 1, wherein the anchor has an uneven outer side.

7. The lead according to claim 6, wherein the uneven outer side of the anchor is at least partially pearly and/or comprises recesses and ridges.

8. The lead according to claim 1, wherein the lead includes at least two anchors.

9. The lead according to claim 8, wherein under conditions where the lead includes the at least two anchors, a first anchor is closest to the electrode section is at a distal part of the lead cable.

10. The lead according to claim 8, wherein at least one of the at least two anchors is at a proximal part of the lead cable.

11. The lead according to claim 9, wherein between the first anchor closest to the electrode section and the electrode section is a free lead cable section without any anchor.

12. The lead according to claim 11, wherein the free lead cable section has a minimum length selected out of a range of approximately 5 mm, approximately 10 mm to 60 mm and/or that the anchor has a length of at least approximately 10 mm or more, or approximately 60 mm or more.

13. The lead according to claim 1, wherein the lead comprises a strain relief loop.

14. The lead according to claim 1, wherein the lead is a spinal cord neurostimulation lead, wherein the electrode section is an electrode paddle with an array of electrodes, and wherein lead is configured to be implanted in vicinity of the cervical or thoracic or lumbar or sacral section of the spinal cord.

15. The lead according to claim 1, wherein the electrode section of the lead comprises at least one anchor and/or is directly connected to at least one anchor.

16. A method of fixing a lead for neuromodulation and/or neurosensing, wherein the lead comprises at least one lead cable, at least one electrode section, and at least one electrode placed in the electrode section, and wherein the lead further comprises at least one anchoring section, wherein the method comprises the steps of:
placing the lead at an implantation site of a subject;
fixing the lead at the implantation site by means of a fixation instrument being engaged with a surrounding tissue of the subject and engaged with the at least one anchoring section.

17. The method of claim 16, wherein the at least one anchoring section comprises a sleeve or sleeve-like section on an outer part of the at least one lead cable, where the sleeve or sleeve-like section forms an outermost part of the lead cable; and
wherein the at least one anchoring section is overmolded over and/or is part of the outer part of the lead cable.

18. The method of claim 16, wherein the at least one anchoring section is at least partially made of one or more of a thermoplastic material and/or an elastomeric material.

19. The method of claim 16, wherein the at least one anchoring section has an uneven outer side, and wherein the uneven outer side is at least partially pearly and/or comprises recesses and ridges, and wherein the method includes engaging the fixation instrument with the surrounding tissue and the at least one anchoring section via winding the fixation instrument around the ridges and where the fixation instrument is held in the recesses.

20. The method of claim 16, wherein the at least one electrode section of the lead is directly connected to the at least one anchoring section.
